(19) United States
(12) Patent Application Publication
(10) Pub. No.: US 2007/0045144 A1
(43) Pub. Date: Mar. 1, 2007

(54) Packaging component for personal care articles

(75) Inventors: Katherine Carol Wheeler, Menasha, WI (US); Teresa Marie Zander, Bonduel, WI (US); Kristi Jo Bryant, Appleton, WI (US); Rebecca Suzanne Walter, Hortonville, WI (US)

Correspondence Address: KIMBERLY-CLARK WORLDWIDE, INC., 401 NORTH LAKE STREET, NEENAH, WI, 54956, US

(73) Assignee: Kimberly-Clark Worldwide, Inc.

(21) Appl. No.: 11/216,397

(22) Filed: Aug. 31, 2005

(51) Int. Cl. A61B 19/02; B65D 73/00

(52) U.S. Cl. 206/494; 206/440

(57) ABSTRACT

The present invention provides a discreet package for absorbent personal care articles. A discreet package containing absorbent personal care articles of the present invention is a flexible package. The flexible package has a packaging component having one or more side panels, a bottom panel and a storage compartment. The side panel has a top edge, which forms an opening which is closable. This side panel may be connected, directly or indirectly to the bottom panel or the bottom panel may be formed from the same web in which the side panel is made. Each of the side panel and the bottom panel have an exterior surface and an interior surface, and the interior surface of the side panel and the interior surface of the bottom panel define the storage compartment, wherein the storage compartment has sufficient volume to hold at least two absorbent personal care articles. Positioned within the storage compartment is a plurality of absorbent personal care articles. To provide discretion to the packaging, the exterior surface of the side panel is devoid of any indicia which tend to indicate that the flexible packaging component has at least one absorbent personal care article placed within said storage compartment of the package.

### FIELD OF THE INVENTION

The present invention relates generally to a packaging component for personal care products, and in particular, to a packaging component for personal care products which provides a greater degree of privacy and discreetness to consumers and users of the personal care products.

### BACKGROUND OF THE INVENTION

Absorbent personal care articles are generally known in the art as products of a personal hygiene or health care nature. Such products include, for example, incontinence articles, diapers, feminine hygiene products, and the like. These absorbent personal care articles have been generally provided to users in containers such as paper containers, generally in the form of a box or carton, or soft side packages such as polymer film bags. These products are typically packaged in a manner such that there is no question to users and non-users as to what the package contains. As a result, the consumer or purchaser of absorbent personal care articles is not provided with a discreet way to purchase the absorbent personal care article. This could lead to embarrassment, anxiousness and a traumatic experience for the purchaser. This is especially the case when the purchaser happens to see someone known to the purchaser during the time between when the package is removed from the shelf or merchandising rack in a store and the time the package is placed into a sack or bag at the point of purchase. Many of the current packages of personal care articles provide no means for discretion.

The current packages for these personal care articles always have indicia on most, if not all, sides of the packages which clearly indicate the contents of the package. As a result of these indicia, the packages with the absorbent personal care articles are often stored in closed storage areas, such as cabinets and drawers, to effectively hide the personal care articles from the sight of others. However, when these items are placed in closed storage, it is often easy for users to forget to use the products, on a daily basis or when the product is needed, especially when the user is pressed for time. Often the absorbent personal care articles are stored near the place of use, which is usually a place where privacy is available, such as a bathroom or bedroom. Further, in many older homes, and relatively cramped living quarters, such as college dorms, small apartments and the like, closed storage in bathrooms or bedrooms is many times not available or is available on a limited basis. As a result, many times the absorbent personal care articles must be stored in the open and in plain view for others, other than the user, to see. This can lead to embarrassment or unnecessary distress for the user of these products, especially when the user must share a space, such as a bathroom, with a nonuser, including guests. Many times, users of the absorbent personal care articles remove the personal care articles from the packaging and place the articles in a more discreet container.

There is a need in the art to provide a discreet package of absorbent personal care articles for purchasers and users of absorbent personal care articles, which provides discretion during the process of purchasing the absorbent personal care articles and after the absorbent personal care articles are placed in storage in the home or other living quarters of the user.

### SUMMARY OF THE INVENTION

Generally stated, the present invention provides a solution to the problem of discreetness of the packaging component for absorbent personal care articles. Provided by the present invention is a discreet flexible package containing absorbent personal care articles. The flexible package has a packaging component having at least one side panel, a bottom panel and a storage compartment. The at least one side panel has a top edge, which forms an opening which is closable. This at least one side panel may be connected, directly or indirectly, to the bottom panel or the bottom panel may be formed from the same web in which one of the side panels is made. Each of the at least one side panel and the bottom panel have an exterior surface and an interior surface, and the interior surface of the at least one side panel and the interior surface of the bottom panel define the storage compartment, wherein the storage compartment has sufficient volume to hold at least two absorbent personal care articles. Positioned within the storage compartment is at least two absorbent personal care articles. To provide discretion to the packaging, the exterior surface of the at least one side panel is devoid of any indicia which tend to indicate that the flexible packaging component has at least one absorbent personal care article placed within said storage compartment of the package.

With the at least one side panel devoid of any indicia indicating the contents of the package, the package of the present invention, with its contents of absorbent personal care articles, may be left in plain sight without clearly indicating the contents of the package to the user or the non-user. As a result, the absorbent personal care articles do not have to be removed from the package to provide discreetness. In addition, by not having indicia on the at least one side panel, a user of the absorbent personal care articles is provided with a discreet means to purchase a supply of the absorbent personal care articles in a retail store. As a result, the user is less likely to have an embarrassing, anxious or traumatic experience during the purchase of the absorbent personal care articles.

### BRIEF DESCRIPTION OF THE DRAWINGS

FIG. 1 shows a cut-away front perspective view of a representative package of the present invention in a closed position.

FIG. 2 shows a cut-away front perspective view of a representative package of the present invention in an opened position.

FIG. 3A shows a possible cross-section of a lower portion of the package prior to loading with the product.

FIG. 3B shows a possible cross section of a lower portion of the package after loading with absorbent personal care articles.

FIG. 4A shows an enlarged view of a possible closure mechanism for the opening of the package.

FIG. 4B shows an enlarged view of a possible closure mechanism for the opening of the package.

FIG. 4C shows a cross-section view of a possible closure mechanism for the opening of the package.

FIG. 4D shows a cross-section view of an alternative closure mechanism for the opening of the package.

FIG. 5 shows a possible configuration for a tamper evident seal.

FIG. 6A shows a possible configuration for an absorbent personal care article placed in the wrapper component of the present invention.

FIG. 6B is a perspective view of a product component in a partially folded configuration.

### DEFINITIONS

It should be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof.

It should be understood that the term “personal care product” or “personal care article” as used herein refers to any article used to control bodily fluids, and includes “absorbent products,” which refers to any article configured to absorb and retain bodily exudates, including urine, bowel movements, blood and menses, and includes such a product in a packaged and unpackaged configuration. As such, “personal care articles” as used herein, includes without limitation, diapers, child toilet training pants, adult incontinence garments, male incontinence products, tampons, vaginal suppositories, pantiliners, pads, sanitary napkins, tissues, wipes, etc. For example, personal care articles include, without limitation, Poise® feminine care articles, including pantiliners and pads, and Kotex® feminine care articles, including sanitary napkins, tampons and liners, all available from Kimberly-Clark Corporation, Neenah, Wis.

As used herein, the term “connected” is intended to mean that two or more members are directly or indirectly connected to one another. When two or more members are directly connected to one another, it is meant that the two members are in direct contact with one another, without an intervening member or structure. When two or more members are indirectly connected to one another, it is meant that the two members are not in direct contact with one another, and may have an intervening member or structure between the two or more members connected to one another.

The phrase “devoid of any indicia which tends to indicate that the package has at least one absorbent personal care article” is intended to mean that the presence of words, symbols, pictures and other marks which would suggest the possible presence of absorbent personal care article within the package are not present on the defined walls and panels. Examples of indicia intended to be excluded include, brand names, words or phrases normally associated with absorbent articles, such as “liners”, “pantiliners”, “tampons”, “sanitary napkins” and the like, or symbols or pictures which depict such absorbent personal care articles. It is noted that this phrase is not intended to exclude information or indicia, which does not imply the presence of an absorbent personal care article, for example, but not limited to, flowers, patterns, or pictures not associated with absorbent personal care articles, and the like.

As used herein, the phrase, “line of weakness” means an area of a material that promotes or enhances separation of a single or unitary structure into at least two structures, or an area of material that promotes or enhances bending or folding without separation.

### DETAILED DESCRIPTION OF THE INVENTION

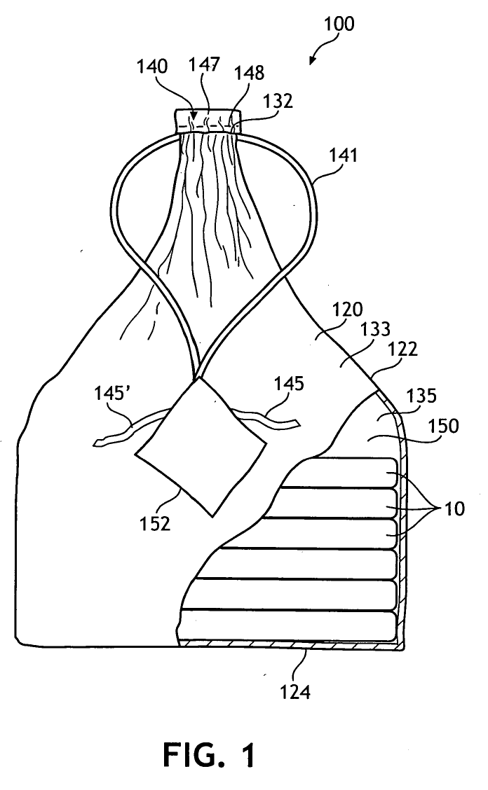

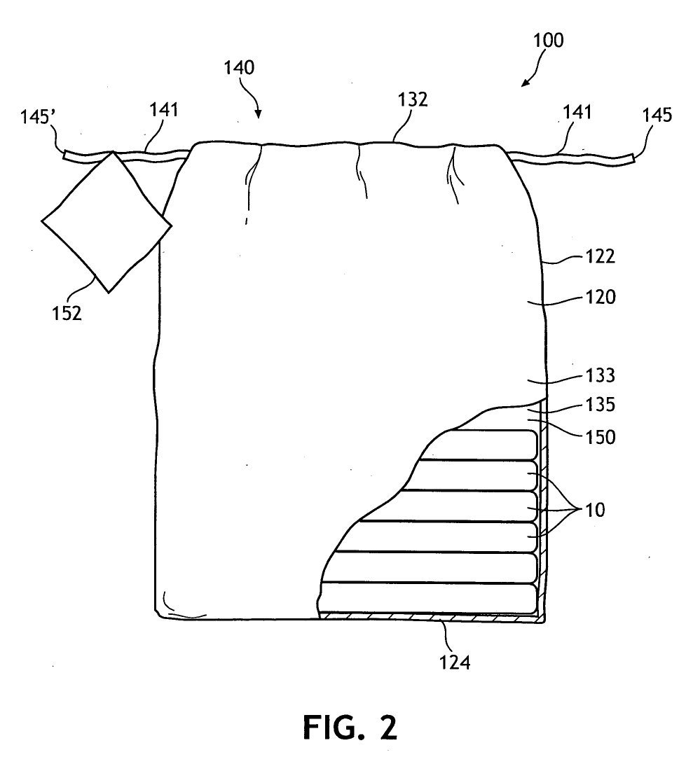

The discreet flexible package of the present invention is designed to transport and hold absorbent personal care articles. To gain a better understanding of the present invention, attention is directed to the Figures. Turning to FIG. 1 and FIG. 2, the flexible package of absorbent personal care articles 100 has a flexible packaging component 120 having one or more side panels 122, a bottom panel 124 and a storage compartment 150. The side panel has a top edge 132, which forms an opening 140 which is closable. This side panel 122 may be connected, directly or indirectly, to the bottom panel 124 or the bottom panel 124 may be formed from the same web in which the side panel 122 is made. Each side panel 122 and the bottom panel 124 have an exterior surface 133 and an interior surface 135, and the interior surface 135 of the side panel 122 and the interior surface 135 of the bottom panel 124 define the storage compartment 150, wherein the storage compartment 150 has sufficient volume to hold at least two absorbent personal care articles. Positioned within the storage compartment 150 is a plurality of absorbent personal care articles 10. To provide discretion to the packaging, the exterior surface 133 of the side wall 122 is devoid of any indicia which tend to indicate that the flexible packaging component 120 has one or more absorbent personal care articles placed within said storage compartment 150 of the package 100.

In one embodiment of the present invention, the package component 100 is devoid of any indicia on exterior surface 133 of the side panels 122 and the bottom panel 124. However, it is not necessary for the exterior surface 133 of the bottom panel 124 to be devoid of the indicia which tend to indicate the presence of one or more absorbent personal care articles. With the exterior surface of the side panels 122 and optionally the bottom panel 124 being devoid of any indicia indicating the contents of the package are absorbent personal care articles, it is difficult for a person not knowing what the contents of the package are to determine the contents of the package, without opening the package. As a result, the package, with its contents of absorbent personal care articles may, discreetly be left in the open, i.e. in plain sight for others to see, without clearly indicating the contents of the package 100. Likewise, during the period from when the package is removed from the shelf by a purchaser, to the time it is bagged at the point of purchase, the fact that the side panel 122 and optionally the bottom panel 124 are devoid of indicia which tend to indicate that the package contains absorbent personal care articles, the package of the present invention provides discretion for the purchaser of the package of absorbent personal care articles. The discretion provided by the package of the present invention allows users and non-users to purchase the absorbent personal care articles without the stigma, embarrassment and anxiety often associated with purchasing absorbent personal care articles.

The flexible packaging component 120 making up the package 100 may be made from any flexible material. The package 100 may be made from any one of a wide variety of materials that are known in the art to be sufficiently flexible to accommodate the desired number of absorbent personal care articles and have sufficient strength to hold and contain the absorbent personal care articles within the package without breaking and without excessive bulging or stretching of the material. Materials include, but are not limited to, polymeric plastic films, foils, paper, paper composites, fibrous webs and the like, or a combination thereof.

Suitable materials can be made from polymeric materials such as polyethylene, polypropylene, polyester, nylon, and the like, as well as any combination thereof. Plastic film materials include, for example, a low density polyethylene (LDPE) film, a LDPE/LLDPE (linear low density polyethylene) film laminate, a LDPE/MDPE (medium density polyethylene) film laminate, a LDPE/HDPE (high density polyethylene) film laminate or the like. In addition, films made from a polyethylene/polypropylene combination may also be used.

The fibrous webs usable as the flexible packaging component of the present invention include woven, knitted or nonwoven fabrics. By making the package out of these materials, an additional benefit of an added level of discretion may be provided, since the package will take a purse-like or sachet-like appearance. In addition, fibrous web material tends to relay a message of comfort to the user, which is not relayed in plastic film packages and offer a degree of uniqueness. From a standpoint of cost, nonwoven webs are desired over the woven or knitted fabrics. Suitable nonwoven fabrics include, for example, airlaid nonwoven webs, spunbond nonwoven webs, meltblown nonwoven webs, bonded-carded-webs, hydroentangled nonwoven webs, spunlace webs and the like. Method of manufacturing each of these materials is known in the art. Laminate of these materials may also be used.

Of these nonwoven webs, the fibrous material web may comprise a nonwoven meltblown web. Meltblown fibers are formed by extruding a molten thermoplastic material through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin, et al. Generally speaking, meltblown fibers may be microfibers that may be continuous or discontinuous, are generally smaller than 10 microns in diameter, and are generally tacky when deposited onto a collecting surface.

The fibrous material web may comprise a nonwoven spunbond web. Spunbonded fibers are small diameter substantially continuous fibers that are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spun-bonded nonwoven webs is described and illustrated, for example, in U.S. Pat. No. 4,340,563 to Appel, et al. U.S. Pat. No. 3,692,618 to Dorschner, et al., U.S. Pat. No. 3,802,817 to Matsuki et al., U.S. Pat. No. 3,338,992 to Kinney, U.S. Pat. No. 3,341,394 to Kinney, U.S. Pat. No. 3,502,763 to Hartman, U.S. Pat. No. 3,502,538 to Levy, U.S. Pat. No. 3,542,615 to Dobo, et al., and U.S. Pat. No. 5,382,400 to Pike, et al. Spunbond fibers are generally not tacky when they are deposited onto a collecting surface. Spunbond fibers can sometimes have diameters less than about 40 microns, and are often between about 5 to about 20 microns.

The fibrous material web may also comprise a laminate material such as a spunbond/meltblown/spunbond, or SMS, material. A typical SMS material is described in U.S. Pat. No. 4,041,203 to Brock et al. Other SMS products and processes are described for example in U.S. Pat. No. 5,464,688 to Timmons et al.; U.S. Pat. No. 5,169,706 to Collier et al.; and U.S. Pat. No. 4,766,029 to Brock et al. Generally, an SMS material will consist of a meltblown web sandwiched between two exterior spunbond webs. Such SMS laminates have been available commercially for years from Kimberly-Clark Corporation under marks such as Spunguard® and Evolution®. The spunbonded layers on the SMS laminates provide durability and the internal meltblown layer provides porosity and additional cloth-like feel.

In another embodiment of the present invention, the flexible packaging material 120 may be a laminate of a film layer and fibrous material layer which have been laminated together by any lamination technique known to those skilled in the art. Suitable lamination means include, but are not limited to, adhesives, ultrasonic bonding and thermomechanical bonding as through the use of heated calendering rolls. Such calendering rolls will often include a patterned roll and a smooth anvil roll, though both rolls may be patterned or smooth and one, both or none of the rolls may be heated. Calandering may also be used to place an aesthetic pattern defined in the laminated wrapper material. Examples of these laminates include, for example, spunbond-film laminates (SF), and other such laminates. In one embodiment, the material for the wrapper component made prepared from a film/spunbond laminate material available from Kimberly-Clark Corp, and known as HBSTL (“highly breathable stretch thermal laminate”), and which material is further disclosed in U.S. Pat. No. 6,276,032, the entire disclosure of which is hereby incorporated herein by reference. Other materials such as elastic films and elastic nonwoven webs may also be used to create a chamber which is expandable. Each panel may be prepared from different materials and each panel may be prepared from one or more material types. For example, one panel may be a film and the other panel a nonwoven web. The flexible packaging material 120 may also be a multi-ply material or a single ply material.

From a standpoint of cost, durability and aesthetic value, it is desirable that the fibrous web is a nonwoven web. Of the nonwoven webs listed above, from a durability standpoint, it is desirable that the nonwoven web be a spunbond nonwoven web, or a spunbond containing laminate. One particular spunbond nonwoven web laminate is a SMS (spunbond/meltblown/spunbond) laminate.

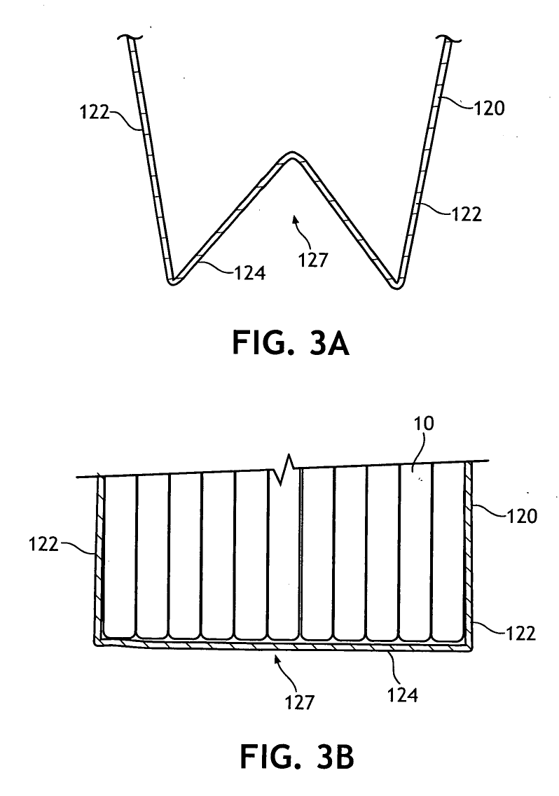

The side panel 122 of the package 100 of the present invention may be prepared from a single piece of one of the materials listed above, or may be prepared from more than one piece of the material. In addition, two or more different types of materials may be used together to form the package to create desired effects, such as aesthetic appeal or physical properties. For example, one panel may be a film material while another may be a fibrous material. An example of possible aesthetic appeal which can be obtained in the present invention by using two or more pieces of material to form the package, is a quilt or patch-work effect, which will further aid in providing discretion for the package. In addition, the bottom panel 124 of the package may be a separate piece of material or can be unitary with the side panels. In one embodiment of the present invention, the bottom panel 124 of the package may be formed by flattening out a gusset 127, as is shown in FIGS. 3A and 3B. In an alternative embodiment of the present invention, the bottom panel 124 may be a separate piece of material which is joined to the one or more side panels 122. In the alternative, the side panel 122 may be joined to the bottom panel 124 indirectly. For example, the side panel 122 may be joined to the bottom panel 124 through one or more other pieces of materials.

Whether the package 100 is prepared from a unitary piece of material or from two or more pieces of material, it will be necessary to join the material or materials together at one or more seams. Any method known to those skilled in the art of joining two pieces of material together may be used. Exemplary joining methods include bonding, sealing, stitching and the like. Suitable bonding and sealing methods include, for example, but are not limited to, adhesive bonding or sealing, bonding or sealing by the application of heat and pressure, ultrasonic bonding or sealing, or any other art known bonding methods. The particular method used to form the seams is not critical to the present invention. However, when the materials are thermoplastic based materials, such as films and nonwoven webs, from the standpoint of ease and speed of production, it is desirable to use pressure and heat or ultrasonic bonding or sealing methods.

The opening 140 of the flexible package 100, which is located at the end of the side panel 122 that is opposite the end attached to the bottom panel 124, should be of a sufficient size to enable a user to open the opening 140 and be able to remove one or more of the absorbent personal care articles 10 from the flexible package 100. The opening 140 may be provided with a closing means 141. Any closing means currently known may be used, including a drawstring, a zipper type mechanism, a button or a series of buttons, a conformable band, an elastic band and the like. The closing means 141 should be sufficient to retain the absorbent personal care articles 10 within the flexible package 100, when not in use, but sufficiently easy to use so that the absorbent personal care articles 10 may be easily removed by a user when desired.

Of the known closure means, an elastic closure means or a draw string closure means are desired from the standpoint of cost and ease of use. When an elastic closure means is used, a single strand of an elastic material may be used or several elastic strands may be used. Optionally, the entire side panel may be prepared from and elastic material. Examples of the closure means includes, for example, an elastic closing member, which may be a single elastic closing member, embedded with the opening end 140 of the side panel 122 or a plurality of elastic members. The elastic strands may be held in place using known techniques, such as stitching, an adhesive means, and placing the elastic strands between layers of the flexible packaging component 120 material.

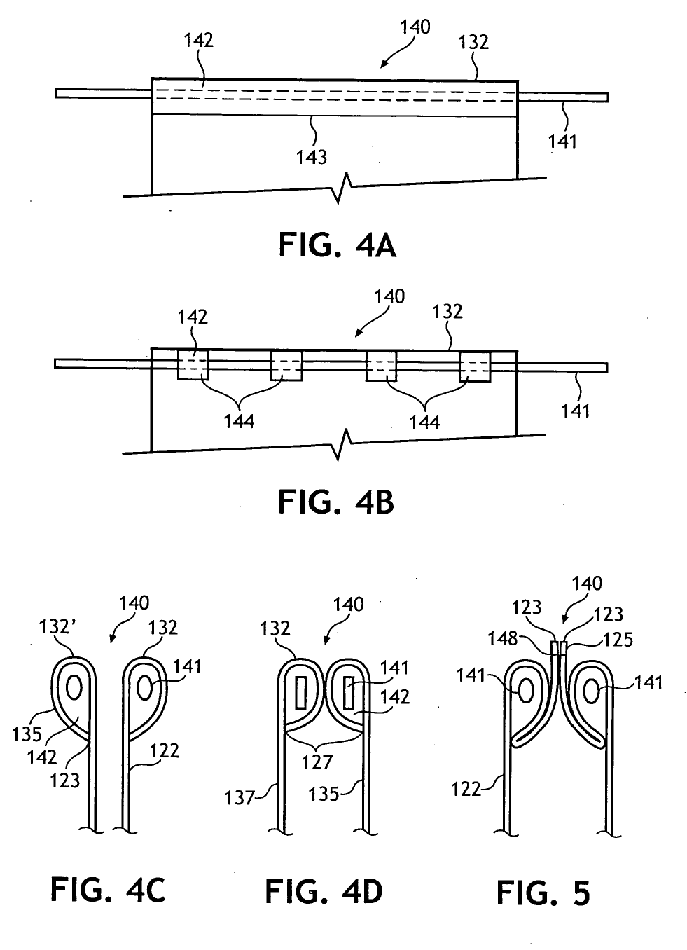

Another closure means include a string or rope-like structure, commonly referred to as a drawstring or a draw cord. The drawstring 141 is shown in FIGS. 1 and 2 and can be made from any material which has sufficient strength to withstand repeated openings and closings. Exemplary drawstring materials include, for example, a string, a twine, a ribbon which can be a woven, a knitted, a nonwoven or film material, or the like. The drawstring may have two free ends 145 and 145′, or the ends may be connected together to form a continuous drawstring. The ends 145 and 145′ may be connected using any known technique, such as tying the ends together, fusing the ends together and the like. Attention is directed to FIG. 4A, FIG. 4B, FIG. 4C and FIG. 4D which each show a possible means for positioning the drawstring 141 in the flexible package 100. In FIG. 4A, shows the drawstring 141 may be placed into a drawstring passage 142, which may be a continuous channel 143. In the alternative, the drawstring 141 may be placed into a drawstring passage 142, which may be a discontinuous channel or a series of shorter channels 144 shown in FIG. 4B. FIG. 4C shows a cross section of the drawstring 141 in the drawstring passage 142, in relationship to the opening 140 for a first possible configuration and FIG. 4D shows a cross section of the drawstring 141 in the drawstring passage 142, in relationship to the opening 140 for a second possible configuration.

The drawstring passage 142 may be made by any method known to those skilled in the art, provided that the drawstring 141 may be pulled in a manner such that the opening 140 of the flexible package may be closed. For example, as is shown in FIG. 4C, the drawstring passage 142 is formed by folding over the end 123 of the side panel 122, partially exposing the interior surface 135 of the side panel and joining the end 123 to the exterior surface 133 of the side panel 122. Any suitable means may be used to join the end 123 to the exterior surface 133 of the side panel 122. Suitable means include, for example, bonding, sealing, stitching and the like. Suitable bonding and sealing methods include, for example, but are not limited to, adhesive bonding or sealing, bonding or sealing by the application of heat and pressure, ultrasonic bonding or sealing, or any other art known bonding methods. The particular method used to form the seams is not critical to the present invention. In the alternative, the drawstring passage 142 may be attached or mounted on the interior surface 135 or exterior surface 133 of the side panel 122 (not shown). In another alternative embodiment of the drawstring closing means, the drawstring passage 142 may be placed on an interior surface 135 of the side panel 122, such that the end 123 of the side panel 122 is folded to come into contact with the interior surface 135 of the side panel 122 and the end 122 is joined to the interior surface 135, as is shown in FIG. 4D.

The flexible package 100 may also be provided with a tamper evident seal 148. One way to provided a tamper evident seal 148 is shown in FIG. 5. In this configuration, the end 123 of the side panel 122 is twice folded, one towards the storage compartment 150 and a second time towards the opening 140 such that the ends 123 extend beyond the top edge 132 of the opening 140, such that there are free flaps 125, 125′ of the flexible package component extending beyond the opening 140. The free flaps 125, 125′ are joined together at a seal area 147 using a suitable means known to those skilled in the art, including those discussed above for joining the side panel 122 to the bottom panel 124. The tamper evident seal 148 may be provided with an opening means, such as a line of weakness 149. One particularly useful line of weakness is a series of perforations located at or very close to the seal area 147, such that the seal may be easily broken by a user.

The package component of the present invention is not restricted in its size. Generally, the package component will be appropriately sized depending on factors such as type of absorbent article placed into the package, and the number of absorbent articles. Larger absorbent articles and higher numbers of absorbent articles require larger packages. For example, a package component containing sanitary napkins would have to be larger than a package component containing pantiliners, assuming that there are similar numbers of each article.

In the present invention, since the flexible package does not contain any indicia of the contents of the package on the exterior surface 133 of at least side panel 122, in order to convey to the consumer the contents of the package prior to purchase and to make the package suitable for sale, the exterior surface of the bottom panel and optionally the exterior surface of the back side wall may be provided with such indicia. The indicia include, for example, branding, any trademarks, bar codes, product descriptions, instructions for the use of the product, instructions for opening the package, manufacture contact information and the like. All such indicia tend to indicate what the contents of the package are. Desirably, the exterior surface of the back side wall does not contain such indicia.

As an alternative to placing all such information on the exterior surface on the bottom, an additional piece of material, hereinafter referred to as the “labeling tag” or “tag” may be use to convey the necessary information to the consumer. This tag could contain all of the necessary indicia mentioned above. The tag should be easily removable from the package by the consumer without damaging the exterior surface of the package. The tag may be held in place on the package by any means, including using an adhesive, tape, or other mechanical mean and the like, provided that the means to hold the additional material to the package does not damage the package when the tag is removed, such that the absorbent personal care articles are visible in the package. The tag may be in the form of a sticker, a booklet, a band of a material and the like. The tag may be placed on one of the side panels 122 or attached to the bottom panel 124.

As is shown in FIG. 1, the tag 152 may be attached to the drawstring 141 which is used as the closure means. It is also noted that the tag 152 may be a single piece of material or several individual pieces. Desirably, the tag 152 is a single piece of material. Generally, the tag 152 is printed or embossed with the indicia, wherein the indicia include items such as branding, instructions, bar code and the like. The tag 152 may be prepared from a wide variety of material including, for example, paperboard, tissue, polymer film, a nonwoven material, sand laminated structures, such as laminated film materials, and the like. Desirably, the tag 152 is material which can be printed with the indicia which tends to indicate the contents of the package. In general, the tag 152 should have at least one other surface without any indicia which indicates the contents of the flexible package so that the user or purchaser may reposition the tag 152 such that the side of the tag which does not have any indicia, which tends to indicate the contents of the container, may be exposed to non-users.

Although the package is devoid on any indicia the exterior surface of the side panel and optionally the bottom panel, which tends to indicate the presence of absorbent personal care articles, does not mean that the package is devoid of all graphics. Pictures and designs unrelated to personal care articles may be present on the exterior surface of the package. In addition, the interior surface of the package, especially the interior surface of the top panel may be imparted with information on how to use the personal care articles placed within the package or other information, such as words of inspiration to the user of the personal care articles. One way of conveying needed information to the user includes placing an insert in the package with instructions on how to use the products.

The absorbent article which may be placed in the interior compartment of the package of the present invention may be any absorbent article including, for example, feminine napkins (sanitary napkins) pantiliners, tampons, interlabial pads, other feminine care articles, adult care articles, child care articles and infant care articles. Although the absorbent article will be further described below in terms of pantiliners or pads, modifications can be easily made by those skilled in the art to use the teachings herein to other absorbent personal care articles.

Absorbent articles such as, for example, feminine care and incontinent absorbent products, generally include a liquid pervious topsheet, a substantially liquid impervious backsheet, and an absorbent core positioned and held between the topsheet and the backsheet. The topsheet is generally operatively permeable to the liquids that are intended to be held or stored by the absorbent article, and the backsheet may be substantially impermeable or otherwise operatively impermeable to the liquids intended to be held or stored. Disposable absorbent articles may also include other optional components or layers, such as liquid wicking layers, liquid distribution layers, barrier layers, and the like, as well as combinations thereof, which may improve the fluid handling and storage properties of the disposable absorbent article. Generally, disposable absorbent articles and the components thereof provide a body-facing surface and a garment-facing surface. As an alternative, the substantially liquid impervious backsheet may be replaced with a liquid pervious backsheet, when the absorbent personal care article is used in conjunction with another liquid impervious layer or article, such as, for example liquid impervious pants. Absorbent articles are exemplified in numerous U.S. patents and patent applications, including, U.S. patent application Ser. No. 10/379,942, entitled “Perimeter Embossing in an Absorbent Article” filed Mar. 4, 2003; U.S. patent application Ser. No. 10/392,116, entitled “Multilayer Absorbent Article, filed Mar. 19, 2003; and U.S. patent application Ser. No. 10/753,974, entitled “Low Profile Absorbent Pantiliner” filed Jan. 7, 2004, all of which are hereby incorporated by reference in their entirety.

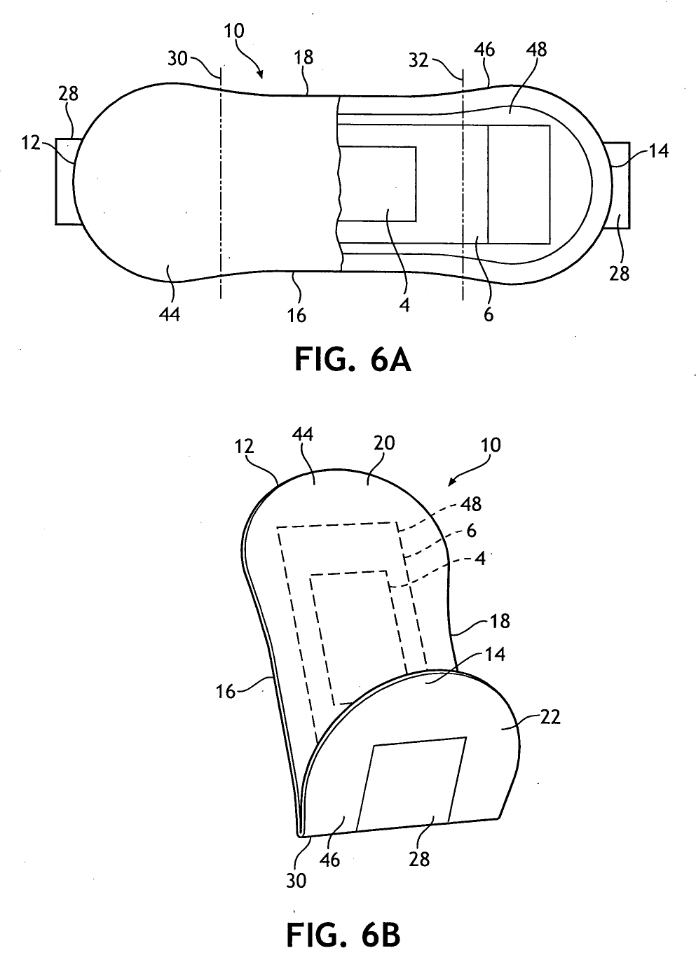

To obtain a better understanding of the absorbent articles which may be present in the packaging component of the present invention, attention is directed to FIGS. 6A and 6B. In FIGS. 6A and 6B, an exemplary absorbent product 10 is shown as including an outer cover 46 (otherwise referred to as a baffle or backsheet), an absorbent core 48, an optional tissue layer 6, an optional distribution layer 4 and a body side liner 44 (also referred to as the top sheet). The absorbent product 10 also has a first side 16 and a second side 18. The first and second sides 16, 18, respectively, are the longitudinal sides of the elongated absorbent product. The sides can be contoured, for example, in a concave shape, or they can be linear. The sides can further include flaps (not shown) that extend laterally outward. Flaps are known in the art and are shown in, for example, U.S. Pat. No. 6,387,084 issued to VanGompel et al., which is hereby incorporated by reference for its discussion of the flaps and flap attachment means and in its entirety. In one embodiment (not shown), one or more elastic elements are disposed along the sides to form a gasket with the body of the user. Elastic sides are known in the art, as is shown in U.S. Pat. No. 6,315,765 issued to Datta et al., which is hereby incorporated by reference for its discussion of the elastic sides and in its entirety. In one embodiment, the elastic elements are disposed between the liner and the outer cover.

The absorbent product 10 has a first body side surface 20 and a second garment side surface 22. Applied to at least a portion of the second garment side surface 22 is a garment attachment adhesive. In various embodiments, the garment attachment adhesive is configured as a single band of adhesive or as two or more spaced apart strips. Alternatively, the garment attachment adhesive includes a swirl pattern of adhesive which encompasses a major portion of the second garment surface 22 of the absorbent article 10.

A release strip 28, also known as a releasable peel strip, is removably secured to the garment attachment adhesive and serves to prevent premature contamination of the adhesive before the absorbent article 10 is secured to, for example, the crotch portion of an undergarment. In various embodiments, the garment attachment adhesive is designed to be secured to the inner crotch portion of an undergarment so as to keep the absorbent product in register with the body of the user. The release strip 28 may extend beyond one or both of the ends 12, 14 of the outer cover, as shown in FIGS. 6A and 6B.

The body side liner or topsheet 44, which is preferably liquid permeable, may be formed from one or more materials. The body side liner or topsheet 44 must be able to manage different body excretions depending on the type of product. In feminine care articles, often the liner or body contacting layer must be able to handle menses and urine. In the present invention, the body side liner or topsheet 44 may include a layer constructed of any operative material, and may be a composite material. For example, the liner or body contacting layer can include a woven fabric, a nonwoven fabric, a polymeric film, a film-nonwoven fabric laminate or the like, as well as combinations thereof. Examples of a nonwoven fabric useable in the body side liner or topsheet 44 include, for example, an airlaid nonwoven web, spunbond nonwoven web, meltblown nonwoven web, a bonded-carded-web, hydroentangled nonwoven webs, spunlace webs or the like, as well as combinations thereof. Other examples of suitable materials for constructing the body side liner or topsheet 44 can include rayon, bonded carded webs of polyester, polypropylene, polyethylene, nylon, or other heat-bondable fibers finely perforated film webs, net-like materials, and the like, as well as combinations thereof. These webs can be prepared from polymeric materials such as, for example, polyolefins, such as polypropylene and polyethylene and copolymers thereof, polyesters in general including aliphatic esters such as polylactic acid, nylon or any other heat bondable materials.

Other examples of suitable materials for the body liner or topsheet 44 are composite materials of a polymeric film and a nonwoven fabric material. The composite materials are typically in the form of integral sheets generally formed by the extrusion of a polymer onto a nonwoven web, such as a spunbond material. In one arrangement, the liner or body contacting layer 44 can be configured to be operatively liquid-permeable with regard to the liquids that the article is intended to absorb or otherwise handle. The operative liquid-permeability may, for example, be provided by a plurality of pores, perforations, apertures or other openings, as well as combinations thereof, which are present or formed in the liner or body contacting layer. The apertures or other openings can help increase the rate at which bodily liquids can move through the thickness of the liner or body contacting layer and penetrate into the other components of the article (e.g. into the absorbent core 48). The selected arrangement of liquid-permeability is desirably present at least on an operative portion of the body side liner or topsheet 44 that is appointed for placement on the body-side of the article. The body side liner or topsheet 44 can provide comfort and conformability, and can function to direct bodily exudates away from the body and toward the absorbent core 48. The body side liner or topsheet 44 can be configured to retain little or no liquid in its structure, and can be configured to provide a relatively comfortable and non-irritating surface next to the body-tissues of a female wearer.

The baffle or backsheet 46 may include a layer constructed of any operative material, and may or may not have a selected level of liquid-permeability or liquid-impermeability, as desired. In a particular configuration, the baffle or backsheet 46 may be configured to provide an operatively liquid-impermeable baffle structure. The baffle or backsheet 46 may, for example, include a polymeric film, a woven fabric, a nonwoven fabric or the like, as well as combinations or composites thereof. For example, the baffle may include a polymeric film laminated to a woven or nonwoven fabric. In a particular feature, the polymeric film can be composed of polyethylene, polypropylene, polyester or the like, as well as combinations thereof. Additionally, the polymeric film may be micro-embossed, have a printed design, have a printed message to the consumer, and/or may be at least partially colored. Suitably, the baffle or backsheet 46 can operatively permit a sufficient passage of air and moisture vapor out of the article, particularly out of an absorbent (e.g. storage or absorbent core 48) while blocking the passage of bodily liquids. An example of a suitable baffle material can include a breathable, microporous film, such as those described in, for example, U.S. Pat. No. 6,045,900 to McCormack et al.

Bicomponent films or other multi-component films can also be used, as well as woven and/or nonwoven fabrics which have been treated to render them operatively liquid-impermeable. Another suitable baffle material can include a closed cell polyolefin foam. Examples of closed cell foams include a closed cell polyethylene foam.

The liquid permeable body side liner 44 and the liquid-impermeable baffle 46 may be peripherally sealed together to enclose the absorbent core 48 to form the absorbent article 10. Alternatively, the body side liner or topsheet 44 can be wrapped around both the absorbent 48 and the baffle or backsheet 46 to form a wrapped pad. The body side liner 44 and baffle 46, and other components of the absorbent product, can be joined for example with adhesive bonds, sonic bonds, thermal bonds, pinning, stitching or any other attachment techniques known in the art, as well as combinations thereof.

The absorbent core 48 is designed to absorb bodily exudates, including menstrual fluid, blood, urine, and other body fluids. The absorbent core 48 may contain one or more layers of absorbent material. The layers can contain similar materials or different materials. Suitable materials for the absorbent core 48 include, for example, cellulose, wood pulp fluff, rayon, cotton, and meltblown polymers such as polyester, polypropylene or coform. Coform is a meltblown air-formed combination of meltblown polymers, such as polypropylene, and absorbent staple fibers, such as cellulose. A preferred material is wood pulp fluff, for it is low in cost, relatively easy to form, and has good absorbency.

The absorbent core 48 can also be formed from a composite comprised of a hydrophilic material which may be formed from various natural or synthetic fibers, wood pulp fibers, regenerated cellulose or cotton fibers, or a blend of pulp and other fibers. In one embodiment of the present invention, the absorbent may be an airlaid material.

In one embodiment, the absorbent core 48 also includes a superabsorbent material, in addition to or in place of the hydrophilic material, which increases the ability of the absorbent core to absorb a large amount of fluid in relation to its own weight. Generally stated, the superabsorbent material can be a water-swellable, generally water-insoluble, hydrogel-forming polymeric absorbent material, which is capable of absorbing at least about 15, suitably about 30, and possibly about 60 times or more its weight in physiological saline (e.g. saline with 0.9 wt % NaCl). The superabsorbent materials can be inserted as particles or in sheet form. The superabsorbent material may be biodegradable or bipolar. The hydrogel-forming polymeric absorbent material may be formed from organic hydrogel-forming polymeric material, which may include natural material such as agar, pectin, and guar gum; modified natural materials such as carboxymethyl cellulose, carboxyethyl cellulose, and hydroxypropyl cellulose; and synthetic hydrogel-forming polymers. Synthetic hydrogel-forming polymers include, for example, alkali metal salts of polyacrylic acid, polyacrylamides, polyvinyl alcohol, ethylene maleic anhydride copolymers, polyvinyl ethers, polyvinyl morpholinone, polymers and copolymers of vinyl sulfonic acid, polyacrylates, polyacrylamides, polyvinyl pyridine, and the like. Other suitable hydrogel-forming polymers include hydrolyzed acrylonitrile grafted starch, acrylic acid grafted starch, and isobutylene maleic anhydride copolymers and mixtures thereof. The hydrogel-forming polymers may be lightly crosslinked to render the material substantially water insoluble. Crosslinking may, for example, be by irradiation or covalent, ionic, Van der Waals, or hydrogen bonding. Hydroxyfunctional polymers have been found to be good superabsorbents for sanitary napkins. Such superabsorbents are commercially available from Dow Chemical, Hoechst-Celanese, and Stockhausen, Incorporated, among others, and are a partially neutralized salt of cross-linked copolymer of polyacrylic acid and polyvinyl alcohol having an absorbency under load value above 25 grams of absorbed liquid per gram of absorbent material (g/g). Other types of superabsorbent materials known to those skilled in the art can also be used.

Additional layers or substrates, including for example, the liquid acquisition and distribution layer 5, also referred to as a surge or transfer layer, and a tissue layer 6 are also incorporated into the absorbent product, for example between the body side liner or topsheet 44 and the absorbent core 48. The distribution layer 5 may be shorter than the absorbent core 48 or have the same length as the absorbent core 48. The distribution layer serves to temporarily hold an insulting fluid to allow the absorbent core sufficient time to absorb the fluid, especially when a superabsorbent material is present. In one embodiment, the absorbent core, transfer layer and other components, such as tissue layers, are free floating (unattached) between the outer cover and the liner, which are secured along only the peripheral edges thereof. Alternatively, the absorbent core, transfer layer and other components are attached to one or both of the outer cover and liner and/or to each other.

The absorbent article may be folded along a pair of fold lines 30, 32 to form a tri-fold configuration, prior to insertion into the wrapper component. In other embodiments, the absorbent article can be bi-folded, flat or rolled. In whichever configuration the absorbent article is in, the absorbent article is placed into a wrapper component of the present invention. Alternatively, a plurality (meaning two or more) of article components can be disposed in a single wrapper component and a plurality of wrapper components, with absorbent article contained therein, can be placed into the flexible package of the present invention.

The absorbent articles of the present invention may be placed into the storage compartment 150 of the packaging component 100 without being further wrapped in an additional component. In the alternative, the absorbent articles may be wrapped in a wrapper component. The wrapper component may have a number of different configurations without departing from the scope of the present invention. These wrapping configurations are well know in the art and any of these known configurations may be used.

The flexible package 100 of the present invention may optionally be provided with a fragrance. Many fragrances have colors associated with the fragrance. For example, the color lavender or purple is often associated with a lavender scent; the color yellow is often associated with a chamomile scent, a daffodil and the like; the color red is often associated with a rose scent and the like; and the color green is often associated with the scents of aloe, wintergreen and the like. Other color and scent combinations may be utilized in the present invention.

At the same time, the first color of the personal care article component, such as the absorbent product, can be coordinated with the first color of one or both of the packaging components. In one embodiment, the second color of the personal care article component, such as the absorbent product, is also coordinated with the second color of one or both of the packaging components. The coordination of the colors is most desirable at distances of less than two feet, such that it is visible to the user of the product. At the same time, the coordination can provide a disguising aspect that is effective for an observer who is greater than 2 to 3 feet away from the product.

The package component of the present invention has a storage compartment which is designed to hold at least two absorbent personal care articles. Typically, the absorbent personal care articles positioned within the storage compartment are of the same type; however, it is not outside the present invention for absorbent personal care articles stored within the chamber to be of different types. For example, the storage compartment may contain pantiliners and tampons, pantiliners and incontinent products, incontinent products and menstrual pads, and so forth. Likewise, the absorbent article within the package component could be of the same type but have different absorbencies, for example, an overnight sanitary napkin and a regular or daytime sanitary napkin. Generally, the package of the present invention contains between 2 and 100 individual absorbent personal care articles, preferably between about 5 and 30 individual absorbent personal care articles.

Those skilled in the art will recognize that the present invention is capable of many modifications and variations without departing from the scope thereof. Accordingly, the detailed description and examples set forth above are meant to be illustrative only and are not intended to limit, in any manner, the scope of the invention as set forth in the appended claims.

1. A discreet flexible package containing absorbent personal care articles comprising:
a flexible packaging component comprising at least one side panel having a top edge, a bottom panel, a storage compartment, and a closable opening, wherein the at least one side panel being connected to the bottom panel, the at least one side panel and bottom panel each have an exterior surface and an interior surface, and the interior surface of at least one side panel and the interior surface of the bottom panel define the storage compartment, said storage compartment having sufficient volume to hold at least two absorbent personal care articles, and the top edge of the at least one side panel forms the closable opening; and
at least two absorbent personal care articles placed within said storage compartment of the packaging component;
wherein the exterior surface of the at least one side wall is devoid of any indicia which tends to indicate that the flexible packaging component has at least one absorbent personal care article placed within said storage compartment of the package.

2. The package of claim 1, wherein the flexible packaging component comprises a nonwoven web.

3. The package of claim 1, further comprising a closing means for the closable opening.

4. The package of claim 3, wherein the closing means for the closable opening comprises a closure chord, which may be manipulated by a user to close the closable opening of the package.

5. The package of claim 4, wherein the closure chord has a first end region and a second end region, and a tag or label placed on the closure cord between the first end region and the second end region.

6. The package of claim 5, wherein the first end region is connected to the second end region.

7. The package of claim 5, wherein the tag or label contains indicia as to the contents of the package.

8. The package of claim 7, wherein the tag or label is removable from the closure cord.

9. The package of claim 3, wherein the closing means for the closable opening comprises an elastic or conformable band, which may be manipulated by a user to open the closable opening of the package.

10. The package of claim 1, further comprising a tag or label which is attached, directly or indirectly to at least one side panel or the bottom panel.

11. The package of claim 10, wherein the tag is attached to at least one side panel.

12. The package of claim 11, wherein the tag or label contains indicia as to the contents of the package.

13. The package of claim 10, wherein the tag is easily removable.

14. The package of claim 1, wherein the at least one side panel comprises between one and eight side panels.

15. The package of claim 1, wherein the at least one side panel and comprises a single continuous panel comprising a single web having a first side edge, a second side edge, a bottom edge, and a top edge, the first side edge is joined to the second side edge, and the bottom edge is joined to the bottom panel and the top edge forms the closeable opening.

16. The package of claim 1, wherein the at least one side panel and the bottom panel comprises a single web, having a first side edge, a second side edge, a bottom region, and a top edge, the first side edge is joined to the second side edge, the bottom region is folded and joined together to form the bottom panel, and the top edge forms the closeable opening.

17. The package of claim 3, wherein the closing means is attached to the exterior surface of the at least one side panel, attached to the interior surface of the at least one side panel, is positioned between the exterior surface and the interior surface of the at least one side panel or a combination of thereof.

18. The package of claim 17, wherein the closing means is positioned partially between the exterior surface and the interior surface of the at least one side wall.

19. The package of claim 1, wherein the package contains between 2 and 100 absorbent articles.

20. The package of claims 19, wherein the package contains between 5 and 30 absorbent articles.
